(19) United States
(12) Patent Application Publication
(10) Pub. No.: US 2005/0154408 A1
(43) Pub. Date: Jul. 14, 2005

(54) Ophthalmic surgical system and method

(76) Inventor: Alexander Dybbs, Cleveland, OH (US)

Correspondence Address: RENNER OTTO BOISSELLE & SKLAR, LLP, 1621 EUCLID AVENUE, NINETEENTH FLOOR, CLEVELAND, OH, 44115, US

(21) Appl. No.: 11/075,610

(22) Filed: Mar. 8, 2005

Related U.S. Application Data
(63) Continuation of application No. PCT/US03/28169, filed on Sep. 9, 2003.
(60) Provisional application No. 60/409,523, filed on Sep. 9, 2002.

(51) Int. Cl. A61F 9/00

(52) U.S. Cl. 606/166

(57) ABSTRACT

A system (20) for making a consistent and uniformly thick resection of the cornea includes a plurality of microkeratomes (22) individually and interchangeably connectable to a controller (24). Each microkeratome (22) has a base that mounts on an eye, a cutting blade that having a cutting edge and an applanator spaced above the cutting edge of the cutting blade. The base has an aperture for receiving an eye therethrough. The microkeratomes (22) have a different size apertures or different blade gap distances between the cutting edge and a bottom of the applanator. The microkeratome (22) is mostly made of plastic, which allows the microkeratome to be used when wet, improving the operation of the microkeratome.

The benefit of the filing date of U.S. Provisional Application No. 60/409,523, filed Sep. 9, 2002 is hereby claimed, and the entire disclosure therein is hereby incorporated by reference.

### FIELD OF THE INVENTION

The present invention relates to an ophthalmic surgical system and method, and more particularly, to a system that includes more than one microkeratome and a method of selecting a microkeratome, as well as a method of preparing a microkeratome for ophthalmic surgery.

### BACKGROUND OF THE INVENTION

A microkeratome is used in a ophthalmic surgical procedure referred to as laser-assisted in situ keratomileusis (LASIK). In LASIK, a surgeon uses the microkeratome on a patient's eye to cut a section of cornea, which is then moved out of the way. Next, the surgeon uses a laser to reshape the corneal layers underneath the removed section to provide vision correction, and the removed section is replaced.

Microkeratomes typically are placed on an eye such that the cornea protrudes through an opening in the microkeratome. A cutting blade then moves across the opening to cut the cornea. Microkeratomes typically are made of metal, particularly surgical stainless steel. In some instances, a part of the microkeratome can be substituted for a different part to provide a different size opening.

### SUMMARY OF THE INVENTION

The present invention provides a new model for predicting microkeratome performance that more accurately explains the interaction of the cornea and the microkeratome, and provides a more accurate prediction of both the diameter and thickness of a corneal resection created by a microkeratome. The results of the model predict that different size apertures provide improved performance for eyes having different geometries. Accordingly, the present invention provides a system and method for selecting different aperture sizes for eyes having different geometry, such as corneal curvature and diameter.

In particular, the present invention provides a system for making a consistent and uniformly thick lamellar resection of a cornea that includes a plurality of microkeratomes individually and interchangeably connectable to a controller. Each microkeratome has a base that mounts on an eye, a cutting blade having a cutting edge, and an applanator spaced above the cutting edge of the cuffing blade. The base has an aperture to receive the cornea of an eye therethrough. The microkeratomes have different size apertures or different blade gap distances. The blade gap distance is the distance between the cutting edge and the bottom of the applanator.

In most microkeratomes, the cutting edge is at a constant height above the upper surface of the base. In the present microkeratome system, the aperture sizes preferably range from a diameter of about eleven millimeters to a diameter of about thirteen millimeters. The blade gap distances range from about one hundred thirty microns to about two hundred twenty microns, and preferably range from about one hundred sixty to about one hundred eighty microns.

The present invention also provides a method for selecting an optimum microkeratome that includes the following steps: providing a plurality of microkeratomes, each having a different aperture size, determining the steepness of the corneal curve, and selecting a microkeratome based on the steepness of the corneal curve, the diameter of the cornea, and the aperture size.

The present invention also provides a nomogram to facilitate selecting an optimum microkeratome from a plurality of microkeratomes. The nomogram has an aperture axis that includes a range of microkeratome aperture sizes, and a corneal curve axis that includes a range of the steepness of corneal curvature. The nomogram also includes a plot of a range of resection diameters for a series of discrete aperture sizes.

The present invention also provides methods of using the microkeratome. For example, the present invention provides a method of preparing a microkeratome for use that includes wetting the microkeratome with a liquid, such as sterile water. The wetting step may include submerging at least a part of the microkeratome in the liquid. The method may also include operating the microkeratome while it is at least partially submerged in the liquid.

The present invention also provides a method of mounting a microkeratome on an eye. The method includes placing a suction ring portion of the microkeratome over an eye such that the eye protrudes through a region bounded by an aperture. The eye cooperates with the custion ring to at least partially define a suction chamber therebetween. Other steps include flooding the eye in the region bounded by the aperture with the wetting liquid and applying a negative pressure to the suction chamber to hold the microkeratome on the eye. The step of flooding the suction chamber may include filling the suction chamber with liquid before applying the negative pressure to the suction chamber.

The foregoing and other features of the invention are hereinafter fully described and particularly pointed out in the claims, the following description and annexed drawings setting forth in detail a certain illustrative embodiment of the invention, this embodiment being indicative, however, of but one of the various ways in which the principles of the invention may be employed.

### BRIEF DESCRIPTION OF THE DRAWINGS

FIG. 1 illustrates an exemplary system for ophthalmic surgery provided by the present invention.

FIG. 2 is a perspective view of a microkeratome of the system of FIG. 1 in relation to an eye.

FIG. 3 is an enlarged perspective view of a part of the microkeratome shown in FIG. 2.

FIG. 4 is a cross-sectional view of a microkeratome mounted on an eye.

FIGS. 5A-5C are progressive schematic illustrations of a corneal lamellar resection by a microkeratome.

FIG. 6 is a nomogram provided by the present invention to facilitate selecting an optimum microkeratome.

FIGS. 7A and 7B illustrate a pre-operative procedure in accordance with the present invention for wetting a microkeratome.

FIG. 8 is a schematic illustration of part of a base of a microkeratome mounted on an eye.

### DETAILED DESCRIPTION

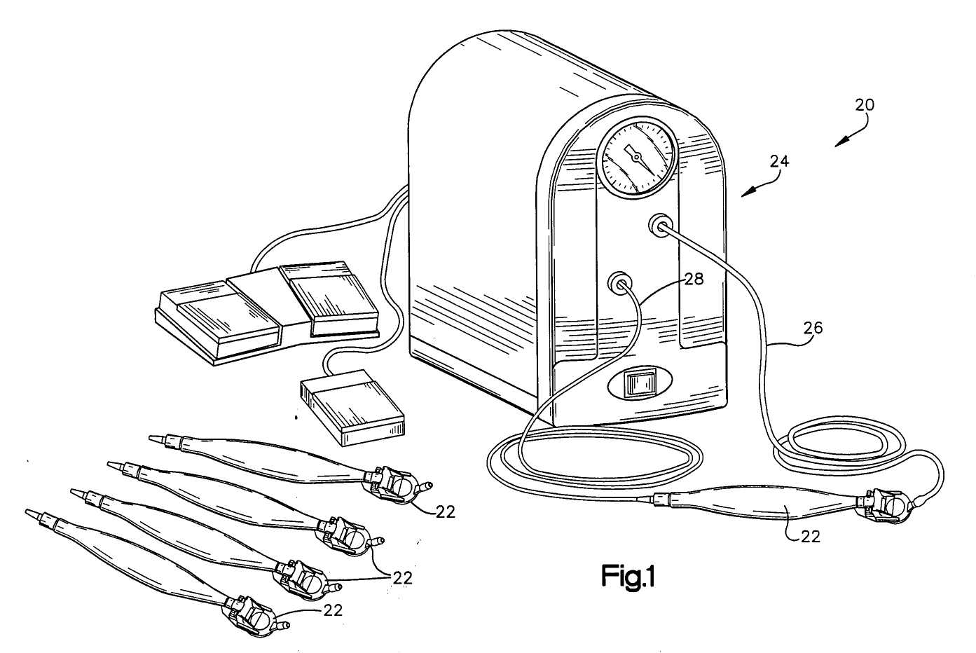

Referring now to the drawings in detail, and initially to FIG. 1, the present invention provides a system 20 for ophthalmic surgery that includes a set of microkeratomes 22, each of which is individually and interchangeably connectable to a controller 24 via a suction tube 26 and a drive cable 28. The different microkeratomes have different size apertures or different blade gap distances, the set of microkeratomes including a plurality of different aperture sizes. The present invention also provides a method of selecting an optimum microkeratome from the set of microkeratomes based on the geometry of the eye for which it will be used, as well as a method of preparing and using the microkeratome.

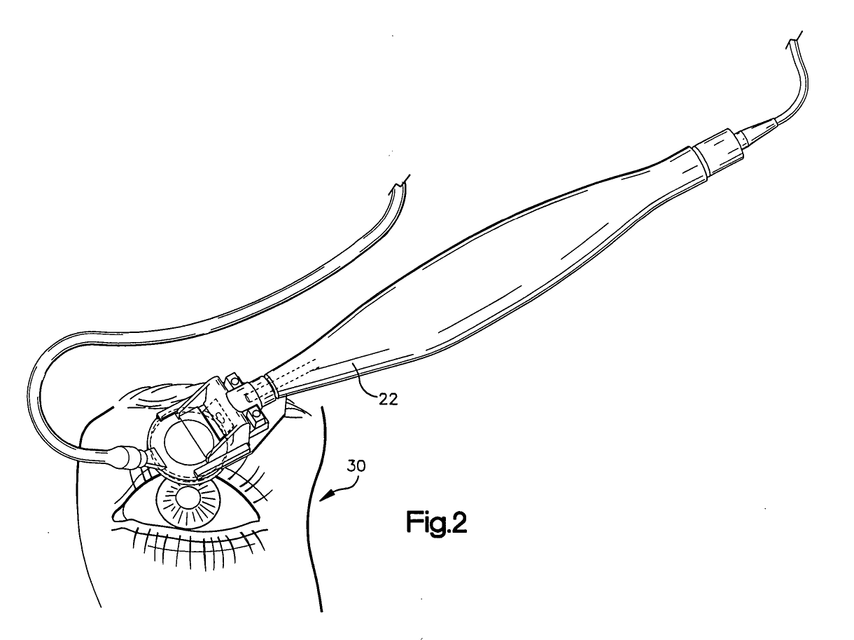

An exemplary microkeratome 22 is shown relative to an eye 30 in FIG. 2. Referring to FIGS. 2-4, the microkeratome 22 has a suction plate or base 32 for mounting on the eye 30 and a cutting blade 34 that moves across the eye 30. The base 32 has an aperture 36 therein through which the cornea 40 of the eye passes to be resected. A suction ring 42 extends from a lower or bottom surface of the base 32. The suction ring 42 is coaxially aligned with the aperture 36 such that when the suction ring is placed on an eye 30, the cornea 40 protrudes through the aperture 36 and extends above an upper or top surface of the base 32. The suction ring 42, the base 32 and the eye 30 cooperate to define a suction chamber 50 therebetween. A conduit 52 in communication with the chamber connects the suction chamber 50 to a suction pump (not shown) in the controller 24 (FIG. 1) to generate suction or negative pressure in the suction chamber 50 to hold the base 32 on the eye 30 during the operation. Preferably, the suction generates at least eighty millimeters of mercury and more preferably approximately ninety millimeters of mercury of pressure in the eye 30. The base 32 thus provides a relatively stable platform for the cutting blade 34.

The cutting blade 34 generally is mounted in a carriage 54 guided for movement across the upper surface of the base 32. In the illustrated embodiment, the carriage 54 holds the cutting blade 34 at an angle of approximately twenty-six degrees with respect to the upper surface of the base. A pair of laterally spaced guides 56 on the base 32 guide the carriage 54 as it moves across the upper surface of the base and across the aperture 36 to resect the cornea 40.

The resection of the cornea 40 may be complete, forming a cap that is completely severed from the cornea, or only partial, forming a flap that is attached to the cornea by an uncut portion or hinge at one side of the resection. The hinge generally has a length of approximately three to four millimeters. Except for the cutting blade 34, the microkeratome 22 preferably is formed of a plastic, and more preferably a clear plastic such as a clear polycarbonate, to give the surgeon a better view of what is happening to the eye 30 as the carriage 54 advances and retracts during the operation.

The carriage 54 in the illustrated embodiment includes an applanator plate 60, sometimes simply referred to as an applanator. The applanator 60 is spaced above the upper surface of the base 32, and also is spaced above and forward of the cutting edge 64 of the cutting blade 34. As the carriage 54 moves the cutting blade 34 and the applanator plate 60 across the aperture 36, the applanator plate flattens the cornea 40 in advance of the cutting blade 34, and the resected portion of the cornea passes between the cutting blade and the applanator 60 as it is cut. The distance between the lower surface of the applanator 60 and the cutting edge 64 of the cutting blade 34 generally is referred to as the blade gap or blade gap distance. This distance is measured along a line perpendicular to the cutting edge of the blade to the bottom of the applanator. The thickness of the resection cut from the cornea is believed to be determined by the blade gap distance. The blade gap distance generally is approximately one hundred thirty to about two hundred twenty microns, preferably approximately one hundred sixty to about one hundred eighty microns.

The purpose of the microkeratome 22 is to make a corneal resection of uniform thickness and also to provide an adequate ablation zone on the cornea under the corneal resection for a laser, for example, to provide the desired corrected vision. Corneas have different geometries that can be and often are measured with a keratometer. Despite differences in corneal geometry, almost all microkeratomes use a single size base with only one aperture size. The aperture generally is designated as approximately eight and a half millimeters or approximately nine and a half millimeters. A nine and a half millimeter designated aperture presumably would cut a nine and a half millimeter diameter resection from the cornea. However, it has been found that a nine and a half millimeter designated aperture generally does not make a nine and a half millimeter resection, and the thickness of the resection may not be what was specified.

The present invention provides a new model to predict microkeratome performance that explains the interaction between the eye and the microkeratome and provides the ability to predict both a lamellar resection diameter and thickness. The results of the model predict that different size apertures provide improved performance. Different apertures may be used for different corneas having different geometries, particularly with regard to diameter and curvature.

If a surgeon uses a large aperture, such as a nine and a half millimeter designated aperture, for example, on a steep cornea, a large volume of cornea is exposed to the applanator plate as it moves across the aperture. The volume can be so large that the forces acting on the applanator plate may force the entire microkeratome to move in a direction away from the eye, resulting in the formation of a thin flap or cap, or even a “buttonhole” in the center of the resected section. Consequently, for a “steep” eye a smaller aperture generally would be preferred.

More particularly, the diameter of the resection is predicted primarily by both the size of the aperture in the base and the curvature of the cornea, larger apertures and steeper corneas both tending to create larger resections. The steeper cornea presents progressively more volume through a given aperture. The combination of a steeper cornea and larger apertures have been associated with a higher probability of thin, irregular, and perforated or buttonholed resections. When the cornea is too steep, especially at the apex where the most corneal volume is encountered, the forces acting on the applanator become markably upward, away from the cornea.

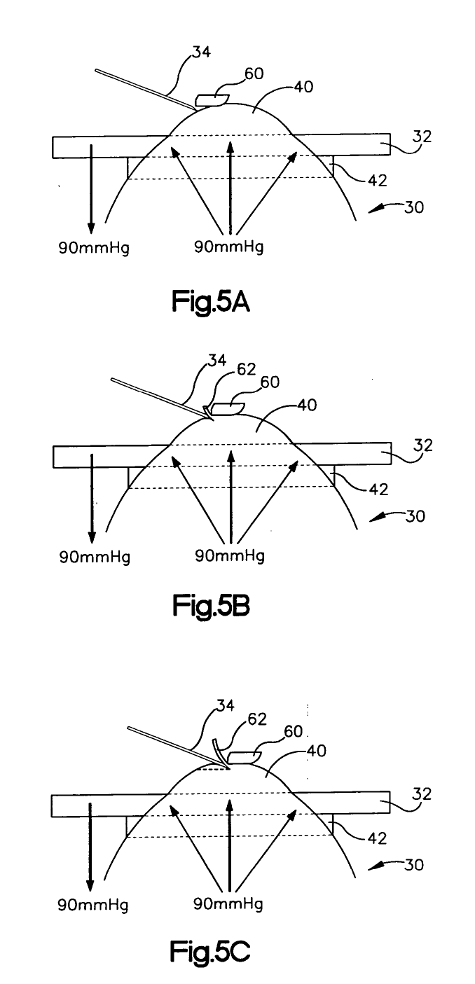

The forces involved are schematically illustrated in FIGS. 5A-5C. The upward forces acting on the microkeratome as the applanator plate 60 moves across the cornea 40 of the eye 30 are distributed to both the base 32 and the carriage (not shown) supporting the cutting blade 34. The entire microkeratome can be forced upward. The movement of the microkeratome generally is not great enough to create a suction break because the tissues on which the suction ring 42 is mounted will stretch somewhat. This upward movement makes the blade cut shallower, sometimes to the point of perforation of the corneal resection 62. Once the applanator plate 60 is beyond the corneal apex in its passage across the cornea 40, the volume of corneal tissue decreases and the upward forces rapidly decline, allowing the microkeratome to move downward and deepening the cut. The cornea 40 does not compress or indent at the approximately ninety millimeters of mercury pressure created by the suction ring 42, particularly due to the fact that the cornea is mostly noncompressible water.

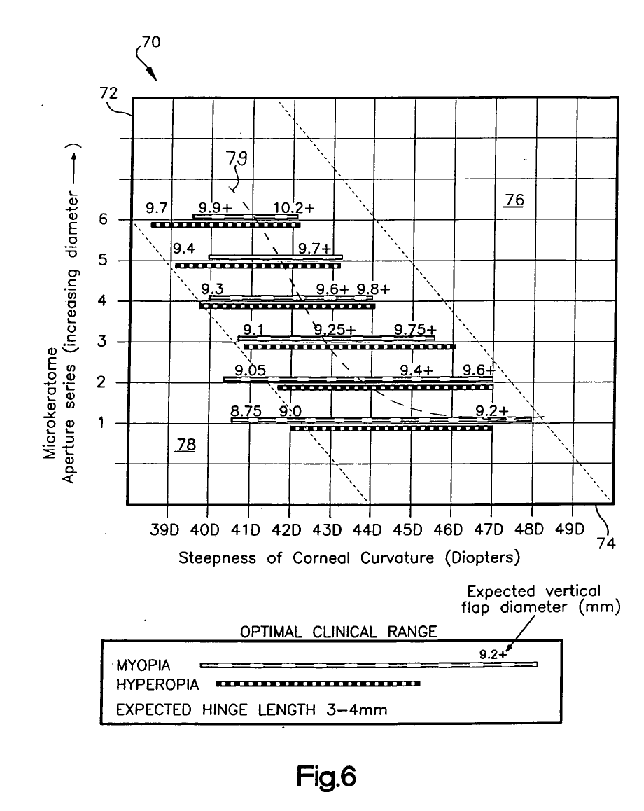

As shown in the nomogram 70 in FIG. 6, the surgeon can determine the optimum series of microkeratome based on the steepness of the corneal curve and the desired diameter of the resection. As mentioned above, the present invention provides a set of microkeratomes 22 (FIG. 1) with different aperture sizes. A surgeon can select one of the different size microkeratomes to provide optimum results for an eye of a given geometry. By determining the steepness of the corneal curvature, preferably the steepest corneal curvature, the surgeon can use the microkeratome aperture series that optimizes the resection size and assures uniform resection thickness for either a myopic or hyperopic eye. In particular, as shown on the vertical axis 72, the microkeratomes are provided in a series of progressively larger aperture sizes ranging from approximately eleven millimeters up to approximately thirteen millimeters in approximately quarter millimeter steps. In addition, the width of the base also varies from approximately seventeen millimeters for a smaller aperture series, to approximately nineteen millimeters for a larger aperture series.

The steepness of the corneal curvature ranges from approximately thirty-eight Diopters to approximately fifty-two Diopters as shown on the horizontal axis 74. For unusually steep corneal curves in combination with large aperture sizes, occasional incidence of thin resection or buttonholes have been found in the region identified by reference number 76. In addition, for shallow corneal curves and small aperture sizes an occasional free cap resection is observed in the region identified by reference number 78 when a flap was desired. For the most part however, the present invention minimizes or eliminates the occurrence of thin resections or buttonholes. An optimum choice can be found near the line 79.

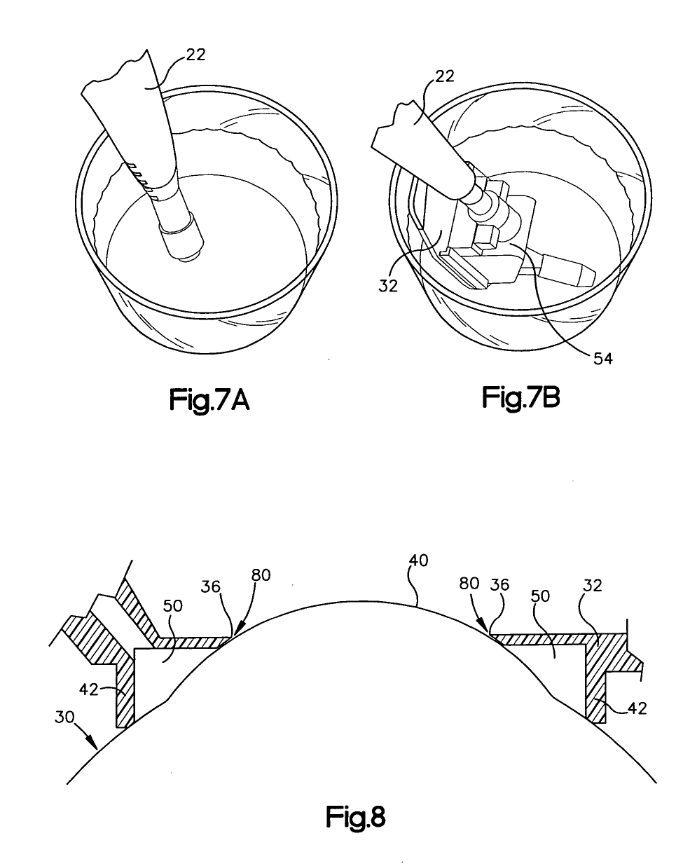

The present invention also provides a method of preparing a microkeratome 22 (FIG. 1) for use. The method includes dipping the microkeratome in a wetting liquid, such as sterile water, prior to the operation. The “wetting liquid,” as is evident from the description of the invention, is a biocompatible liquid that does not come from body fluids. This may include simply dipping the rear portion of the microkeratome 22 in the water (as shown in FIG. 7A) for about five seconds and then submerging at least the forward portion of the microkeratome 22 in the water (as shown in FIG. 7B) prior to the surgery. In dipping the forward portion of the microkeratome 22 (including the carriage 54), it is preferred that the microkeratome be operated and the carriage 54 moved forward and backward across the base 32 several times while submerged to lubricate the moving parts.

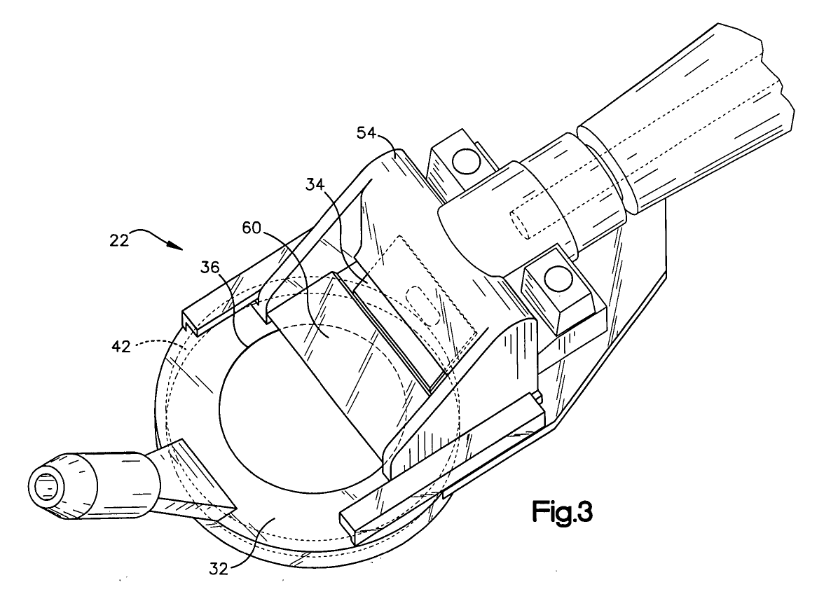

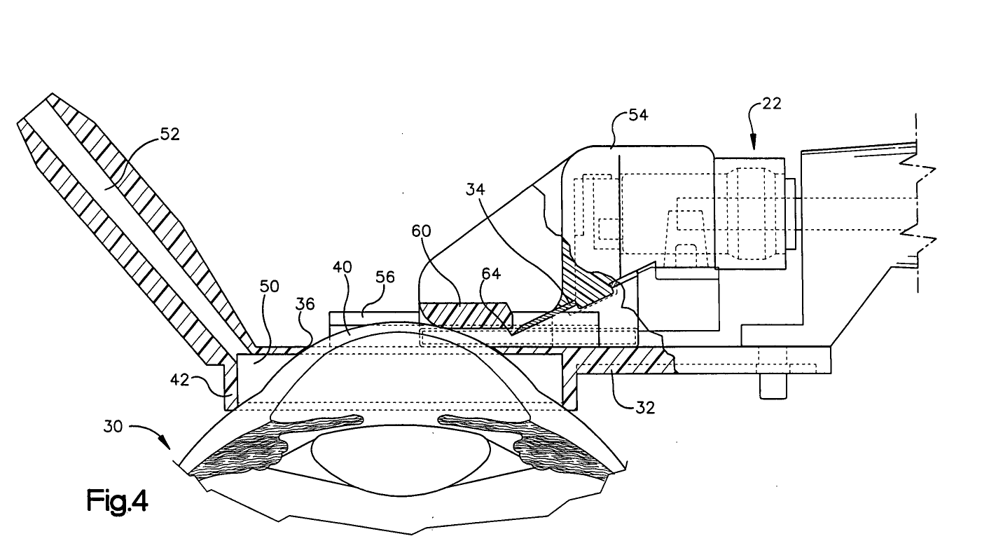

In fact, in accordance with the present invention it is desirable, and indeed preferable, to conduct the entire operation in a wet environment. In particular, referring to FIG. 8, a wetting liquid, such as a sterile balanced saline solution (BSS), is applied to the eye 30 before the base 32 of the microkeratome 22 (FIG. 4) is placed on the eye. Initially, only the suction ring 42 engages the eye 30. The cornea 40 may or may not engage the inner surface of the aperture 36 in the base 32 through which it extends. After the base 32 is gently placed on the eye 30, the surgeon floods the eye with the wetting liquid before suction is applied. The liquid is directed onto the cornea 40 from above and inside the boundary of the aperture 36, which generally does not seal tightly to the cornea 40 before suction is applied. The wetting liquid fills the gap at 80 between the cornea 40 and the sides of the aperture 36 to provide a seal that facilitates the application of suction to create a sealed suction chamber 50. The suction chamber 50 may be completely filled with the wetting liquid to drive out any air. The preferred microkeratome has a clear base 32 that allows the surgeon to visually observe the suction chamber 50 filling and the liquid driving out the air. The liquid-filled suction chamber 50 provides a visual indication to the surgeon when the suction can be applied to hold the base 32 on the eye 30. Upon application of the suction, a clear indication of a good seal on the eye 30 can be observed through the clear base 32 as a blanched portion of the eye. The negative pressure in the suction chamber 50 gently pulls the base 32 of the microkeratome down on the eye 30 or the eye 30 is pulled up into the base 32.

This procedure is gentle on the eye and provides a better seal against the eye. Not only would the portion of the eye in the suction ring 42 not be visible in a metal microkeratome, but the continual exposure to a liquid during the procedure would be anathema to the surgeon using a metal microkeratome because of the risk of corrosion. In addition, the surgeon using metal microkeratome bases often must press down on the base, pressing the suction ring and the sides of the aperture against the eye to obtain a seal while the suction is initially applied. This can lead to increased trauma, including abrasions on the surface of the eye, and can cause increased patient discomfort and recovery time. In contrast, little or no trauma has been observed in procedures using the wet field technique described herein.

The microkeratome is almost completely made of a plastic material, such as polycarbonate, and the liquid provides a lubricating effect between the moving parts. In contrast, since most existing microkeratomes are made of metal, the microkeratome and the surgical area heretofore generally have been maintained in as dry an environment as possible to avoid corrosive effects. Indeed, some metal microkeratomes require use of a synthetic lubricant, contamination of which can lead to complications. Since water and saline solution are biocompatible, the contamination problems associated with synthetic lubricants are eliminated. In addition, because the microkeratome is relatively simple and inexpensive to manufacture, it can be preassembled, sterile, and disposable. Accordingly, the present invention provides a new and improved system and methods for ophthalmic surgery.

Although the invention has been shown and described with respect to certain illustrated embodiments, equivalent alterations and modifications will occur to others skilled in the art upon reading and understanding the specification and the annexed drawings. In particular regard to the various functions performed by the above described integers (components, assemblies, devices, compositions, etc.), the terms (including a reference to a “means”) used to describe such integers are intended to correspond, unless otherwise indicated, to any integer which performs the specified function (i.e., that is functionally equivalent), even though not structurally equivalent to the disclosed structure which performs the function in the herein illustrated embodiments of the invention. In addition, while a particular feature of the invention may have been described above with respect to only one of several illustrated embodiments, such a feature may be combined with one or more other features of the other embodiment, as may be desired and advantageous for any given or particular application.

1. A system for ophthalmic surgery, comprising: a plurality of microkeratomes individually and interchangeably connectable to a controller, each microkeratome having a base that mounts on an eye, a cutting blade having a cutting edge, and an applanator spaced above the cutting edge of the cutting blade, the base having an aperture to receive the cornea of an eye therethrough, wherein the microkeratomes have different size apertures or different blade gap distances, the blade gap distance being the distance between the cutting edge of the cutting blade and the bottom of the applanator.

2. A system as set forth in claim 1, wherein the base has a suction ring extending from a bottom side of the base that is coaxially aligned with the aperture, the suction ring being adapted to mount the base on an eye.

3. A system as set forth in claim 1, wherein the aperture sizes range from a diameter of about eleven millimeters to a diameter of about thirteen millimeters.

4. A system as set forth in claim 1, wherein the blade gap distance is about one hundred thirty microns to about one hundred eighty microns.

5. A system as set forth in claim 4, wherein the blade gap distance is about one hundred sixty microns to about one hundred eighty microns.

6. A system as set forth in claim 5, wherein the blade gap distance for each microkeratome is approximately one hundred sixty millimeters.

7. A system as set forth in claim 1, wherein each microkeratome includes a carriage mounted on a base, the carriage supporting the cutting blade for movement relative to the base.

8. A system as set forth in claim 1, wherein the carriage includes the applanator.

9. A method of preparing a microkeratome for ophthalmic surgery, comprising providing a microkeratome, and wetting the microkeratome with a liquid.

10. A method as set forth in claim 9, wherein wetting includes wetting the microkeratome with at least one of sterile water and sterile saline solution.

11. A method as set forth in claim 9, wherein wetting includes submerging at least a portion of the microkeratome in the liquid.

12. A method as set forth in claim 11, wherein submerging includes submerging at least a portion of the microkeratome in the liquid for about five seconds.

13. A method as set forth in claim 11, further comprising operating the microkeratome while wetting the microkeratome with the liquid.

14. A method as set forth in claim 13, wherein operating includes operating the microkeratome while it is submerged in the liquid.

15. A method of mounting a microkeratome on an eye for ophthalmic surgery, comprising: placing a suction ring portion of a microkeratome over an eye such that the eye protrudes through a region bounded by an aperture, the eye cooperating with the suction ring portion to at least partially define a suction chamber therebetween; flooding the eye in the region bounded by the aperture with the wetting liquid; and applying a negative pressure to the suction chamber to hold the microkeratome on the eye.

16. A method as set forth in claim 15, wherein flooding includes flooding the eye with at least one of sterile water and balanced sterile saline solution.

17. A method as set forth in claim 15, wherein flooding includes flooding the eye until the suction chamber is substantially filled with the liquid.

18. A method as set forth in claim 15, further comprising wetting the eye with the wetting liquid prior to placing the suction ring portion of the microkeratome over the eye.

19. A method as set forth in claim 18, further comprising operating the microkeratome while wetting the microkeratome with the liquid.

20. A method for selecting an optimum microkeratome, comprising providing a plurality of microkeratomes, having respective different aperture sizes;
determining the steepness of the corneal curve; and selecting one of a plurality of microkeratomes based on the steepness of the corneal curve and the aperture size.

21. A method as set forth in claim 20, wherein selecting the microkeratome includes selecting a microkeratome also based on the corneal diameter.

22. A nomogram for selecting an optimum microkeratome from a plurality of microkeratomes, comprising an aperture axis including a range of microkeratome aperture sizes, a corneal curvature axis including a range of steepest corneal curvatures; and a plot of a range of resection diameters for a series of discrete aperture sizes.

23. A nomogram as set forth in claim 22, wherein the plot of resection diameters includes different resection diameters for correcting hyperopia and myopia.
